(19) United States
(12) Patent Application Publication
(10) Pub. No.: US 2005/0142658 A1
(43) Pub. Date: Jun. 30, 2005

(54) Chromosomal saturation mutagenesis

(76) Inventors: Jay M. Short, Rancho Santa Fe, CA (US); Michelle Cayouette, San Diego, CA (US)

Correspondence Address: DIVERSA C/O MOFO S.D., 3811 VALLEY CENTER DRIVE, SUITE 500, SAN DIEGO, CA, 92130, US

(21) Appl. No.: 10/494,875

(22) PCT Filed: Dec. 3, 2002
(86) PCT No.: PCT/US02/38587

Related U.S. Application Data
(60) Provisional application No. 60/336,567, filed on Dec. 3, 2001.

(51) Int. Cl. C12N 15/00

(52) U.S. Cl. 435/325; 435/440

(57) ABSTRACT

In One aspect, the invention provides methods for Chromosomal Saturation Mutagenesis (CSM) comprising generating randomly mutated, overlapping segments for an entire chromosome using error-prone PCR or other techniques, and libraries of nucleic acids made by these methods.

### TECHNICAL FIELD

This invention generally relates to molecular genetics. In one aspect, the invention provides methods for Chromosomal Saturation Mutagenesis (CSM) and libraries of nucleic acids made by these methods. The methods can comprise generating randomly or non-randomly mutated, overlapping segments for an portion of or an entire chromosome, or an entire genome, using error-prone PCR or other techniques. In one aspect, these segments are inserted precisely into a homologous chromosomal locus using a markerless gene replacement technique without addition of exogenous sequences.

### BACKGROUND

Researchers have used UV or chemical methods to introduce point mutations into random locations of chromosomes to create hosts with desired phenotypes. The randomness of these approaches has proven to be both an advantage and a disadvantage because, although no sequence or functional information is needed to effect a desired phenotypic change, it is difficult to identify the mutation(s) that leads to an altered phenotype. This limits the amount of information that can be extracted from these approaches. More importantly, these relatively gross methods concomitantly produce numerous mutations whose phenotypic effects are not always well defined or necessarily desirable.

P1 transduction has been used to transfer a desired mutation generated from a mutagenized strain to a strain with an un-mutated background to minimize potentially deleterious side mutations generated during exposure to the mutagenic agent. However, because the transfer and recombination rate with P1 transduction is low, the mutation must be either itself selectable or a selectable marker must be transduced along with the gene to select cells that contain the desired mutation.

Amore controlled chromosomal mutagenesis is possible using transposable elements. Transposon mutagenesis can precisely map an insertion site of the transposon with minimal effort after a desired phenotype is discovered. This is important because the ability to link genotypic and phenotypic data is critical for understanding the functional genomics of an organism. As a result, transposon mutagenesis offers a tool for evolving bacterial chromosomes and to link structural and functional information in ways that permit the engineering of hosts that are particularly suited to specific applications.

Although transposons may offer advantages over random UV or chemical mutagenesis, there is a limit as to what can be accomplished by simply disrupting a region of the chromosome by transposon insertion. Often, the introduction of single or multiple point mutations is required to achieve a desired phenotypic change. In protein evolution studies, random mutagenesis of a particular gene has been shown to improve the activity, increase the stability, or change the substrate specificity of many enzymes. Techniques are available to generate diverse collections of mutations within cloned genes.

Homologous recombination or mismatch repair systems can be used to introduce specific point mutations into a defined region of a host's chromosome. Although effective, these approaches have proven to be inefficient, slow and labor intensive. In addition, researchers must screen the surviving clones to determine which of the clones harbors the desired mutation. Posfai (1999) Nucl. Acids Res. 27:4409-4415, discusses a method that efficiently introduces a specific mutation into a defined region of an E. coli chromosome in a markerless fashion and selects against those that have not recombined. This technique can be been used to incorporate specific point mutations, small insertion, or deletions.

### SUMMARY

The invention provides methods for Chromosomal Saturation Mutagenesis (CSM). CSM can comprise generating mutated, e.g., randomly mutated or directed mutations, of segments, e.g., overlapping segments, for a part of or an entire chromosome, or an entire genome, using error-prone PCR or other techniques. In one aspect, these segments are inserted precisely into a homologous chromosomal locus. In one aspect, this is done using a markerless gene replacement technique without addition of exogenous sequences.

In one aspect, the Chromosomal Saturation Mutagenesis (CSM) methods comprise the steps of a) dividing up a genome or a chromosome into segments; and b) introducing mutations into one or more segments. All of the segments, all of a chromosome or the entire genome can be mutated. In alternative aspects, the segments are between about 10 and 500,000 base pairs (bp) or more, or, between about 50 and 250,000 base pairs (bp), or, between about 100 and 100,000 base pairs (bp), or between about 200 and 5,000 base pairs (bp). In alternative aspects, the segments are about 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500,3000, 3500, 4000, 4500, 5000, 10,000, 20,000, 30,000, 40,000, 50,000, 60,000, 70,000, 80,000, 90,000, 100,000, 200,000, 300,000, 400,000, 500,000 or more base pairs (bp).

In alternative aspects, the segments can be either overlapped or not overlapped. In alternative aspects the overlaps can be between about 2 and 50 base pairs, or, 2, 3, 4, 5, 6, 7, 8, 9, 10, 11, 12, 13, 14, 15, 16, 17, 18, 19, 20, 25, 30, 35, 40, 45, 50, 55 or more base pairs.

In alternative aspects, the number of mutations to each segment can be between one residue and all of the residues of the segment. The number of mutations to each segment can be anywhere between 1% and 100% of the residues on a segment. For example, number of mutations to each segment can be 1%, 5%, 10%, 15%, 20%, 25%, 30%, 35%, 40%, 45%, 50%, 55%, 60%, 65%, 70%, 75%, 80%, 90%, 95% or 100% of the residues on a segment.

In alternative aspects, the mutations are randomly introduced, or, non-randomly introduced, or, a combination of both. In one aspect, the mutations are introduced by polymerase chain reaction (PCR), such as error-prone polymerase chain reaction (PCR).

In one aspect, the mutated segments can comprise a GSSM library, an SLR library, or a TGR (Tunable Gene Reassembly) library at one or more segments. Thus, in different aspects the concentration of mutations can be a range, e.g. in GSSM there can be 64 possibilities in length of 3 bases.

In alternative aspects, the mutations are in open reading frames (ORFs) or non-ORF's, coding regions or non-coding regions, and mixtures thereof.

In alternative aspects, the organism to be mutagenized or the origin of the nucleic acid segment to be mutagenized can be a bacterium, e.g., E. coli or other any other microbe or prokaryote, or a eukaryote. The mutagenized organism can be any host, e.g., Streptomyces, Pseudomonas, Arabidopsis, including any host used for recombinant expression.

In one aspect, the a part of or an entire chromosome or genome can be mutagenized. For example,1%, 5%, 10%, 15%, 20%, 25%, 30%, 35%, 40%, 45%, 50%, 55%, 60%, 65%, 70%, 75%, 80%, 90%, 95% or 100% of a genome or a chromosome can be mutagenized or can comprise of one or more segments to be mutagenized. In alternative aspects, a portion of the genome (or chromosome) to be mutagenized (comprising of one or more segments to be mutagenized) is at least approximately 100 bp, 500 bp, 1000 bp, 10³ bp, 10⁴ bp, 10⁵ bp, 10⁶ bp, 10⁷ bp, up to an entire chromosome or genome; for example, the E. coli genome is about 3,600 kb, and other genomes are much larger.

In one aspect, the mutated nucleic acid segments are introduced to a homologous chromosome in vitro. In one aspect, the mutated nucleic acid segments are introduced to a homologous chromosome in vivo. In one aspect, the mutated nucleic acid segments are introduced together with a selection marker, e.g., an antibiotic selection marker. In alternative aspects, the antibiotic selection marker is ampicillin resistance. In one aspect, the selection marker involves a secreted product that, if the cells are grown in suspension and not on a plate, can protect other cells without the marker. In one aspect, the secreted selection marker (e.g., resistance marker) is an antibiotic selection marker, such as an ampicillin selection marker. Exemplary selection markers used in the methods of the invention include beta lactam antibiotics, e.g., semisynthetic penicillins, such as amoxycillin, ampicillin, methicillin, carbenicillin. Exemplary selection markers used in the methods of the invention also include tetracyclines, chloramphenicol, the macrolides (e.g. erythromycin) and the aminoglycosides (e.g. streptomycin). Exemplary selection markers used in the methods of the invention also include nalidixic acid, quinoline, rifamycins, sulfonamides (e.g. Gantrisin) and Trimethoprim.

In alternative aspects, the cells are grown at lower temperatures than 37° C., 30° C., 25° C. or 20° C. In different aspects, the cells can be grown in suspensions or plates.

In alternative aspects, after insertion of the mutated segment (e.g., chromosome, or, entire genome) into a host, altered genotypes and/or phenotypes are selected for. In alternative aspects, the selection criteria include: a) increased or decreased expression of a biomolecule such as a small molecule or a protein (this can be exogenous or endogenous); b) inactivation or inhibition of a protease, so that a protein that is susceptible to degradation by that protease can be expressed at higher levels; c) inhibition or alteration of a “feedback inhibition mechanism” in a cell; d) alteration of a set of genes or gene products; e) alteration of a metabolic pathway (this can re-direct metabolites differently); f) alteration of a cell from being to not being an auxotroph, and vice versa as well as both (an organism, such as a strain of bacteria, that has lost the ability to synthesize certain substances required for its growth and metabolism as the result of mutational changes); g) alteration of a cell from being to not being a prototroph, and vice versa as well as both (back and forth) (having the same metabolic capabilities and nutritional requirements as the wild type parent strain, e.g. prototrophic bacteria).

The invention provides methods for mutating a nucleic acid sequence comprising the following steps (a) providing segments of a chromosome or part of a chromosome; (b) introducing one or more mutations into one or more of the segments; and (c) reinserting the mutated segments into a homologous chromosome with a markerless gene replacement technique.

In one aspect, the chromosome of step (a) comprises an entire chromosome or an entire genome.

In one aspect, the chromosome is a bacterial chromosome, such as an E. coli chromosome. The chromosome can be a yeast, plant, insect or mammalian chromosome, such as a human chromosome.

In one aspect, the segments are overlapping. The segments can be overlapping by 2, 3, 4, 5, 6, 7, 8, 9, 10, 11, 12, 13, 14, 15, 16, 17, 18, 19, 20, 25, 30, 35, 40, 45, 50 or 55 base pairs. In one aspect, the segments are not overlapping. The segments can be about 5, 10, 20, 30, 40, 50, 60, 70, 80, 90, 100, 150 or 200 base pairs in length. The segments can be about 250, 300, 350, 400, 450, 500, 550, 600, 650, 700, 750, 800, 850, 900, 950, 1000, 1500, 2000, 2500, 3000, 3500, 4000, 4500, 5000 base pairs in length. The segments can be between about 10 and 500,000 base pairs, or, between about 50 and 250,000 base pairs, in length.

In one aspect, the mutations are randomly introduced. In one aspect, the mutations are non-randomly introduced. The mutations can introduced by polymerase chain reaction (PCR), such as by error-prone polymerase chain reaction (PCR). The error-prone polymerase chain reaction (PCR) can be a Taq-based error-prone PCR.

In one aspect, the mutated segments can comprise a GSSM library, an SLR library, or a TGR (Tunable Gene Reassembly) library at one or more segments.

In one aspect, the mutations are introduced into polypeptide open reading frames. The mutations can be introduced into non-coding sequences.

In alternative aspects, 1%, 5%, 10%, 15%, 20%, 25%, 30%, 35%, 40%, 45%, 50%, 55%, 60%, 65%, 70%, 75%, 80%, 90%, 95% or 100% of a genome or a chromosome is mutagenized.

In one aspect, the mutated nucleic acid segments are introduced to a homologous chromosome in vitro. In one aspect, the mutated nucleic acid segments are introduced to a homologous chromosome in vivo.

In one aspect, the method further comprises inserting the mutated segments into a host cell comprising the homologous chromosome. The mutated nucleic acid segments can be introduced into the host cell with a selectable marker. The selectable marker is an antibiotic selection marker, such as ampicillin, a beta lactam antibiotics, a semisynthetic penicillin, amoxycillin, ampicillin, methicillin, carbenicillin, tetracycline, chloramphenicol, a macrolide, erythromycin, an aminoglycoside, streptomycin, nalidixic acid, quinoline, rifamycin, sulfonamide, Gantrisin or Trimethoprim.

In one aspect, the method further comprises selecting a host cell comprising an altered genotype. In one aspect, the method further comprises selecting a host cell comprising an altered phenotype.

In one aspect, the mutated segments are cloned into a vector before insertion into the host cell. The mutated segments can be inserted into the host cell by any means, e.g., electroporation, infection, transformation or transfection.

The invention provides libraries of mutated nucleic acid sequence made by a method of the invention, e.g., a method comprising the following steps: (a) providing segments of a chromosome or part of a chromosome; (b) introducing one or more mutations into one or more of the segments; and (c) reinserting the mutated segments into a homologous chromosome with a markerless gene replacement technique.

The invention provides cells comprising a library of mutated nucleic acid sequences, the library made by a method of the invention, e.g., a method comprising the following steps: (a) providing segments of a chromosome or part of a chromosome; (b) introducing one or more mutations into one or more of the segments; and (c) reinserting the mutated segments into a homologous chromosome with a markerless gene replacement technique.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 schematically illustrates an exemplary CSM method of the invention.

FIG. 2 shows a table comparing exemplary PCR based mutagenesis methods used in the methods of the invention, as described in detail in Example 1, below.

FIG. 3 schematically illustrates an exemplary markerless replacement method used in the methods of the invention.

FIG. 4 is an illustration of a 1% agarose, 1×TAE gel with data demonstrating the recombination of a mutated version of lamB into a chromosome using an exemplary method of the invention, as described in detail in Example 2, below.

FIG. 5 is an illustration of the gross recombination site mapping, as described in detail in Example 2, below.

Like reference symbols in the various drawings indicate like elements.

### DETAILED DESCRIPTION

The invention provides methods for Chromosomal Saturation Mutagenesis (CSM). CSM can be used to generate pools of mutant alleles and to generate diverse and ordered genomic libraries. CSM can be used for large-scale genome evolution.

CSM comprises generating mutations over overlapping segments for an entire chromosome using error-prone PCR or other techniques. The errors can be generated by random mutation or by directed mutation. In one aspect, these segments are inserted precisely into a homologous chromosomal locus. The insertion can be by using a markerless gene replacement technique without addition of exogenous sequences. The methods can generate mutations over a segment of a chromosome, or, an entire chromosome.

The methods can generate mutations over all or part of a chromosome of any origin, e.g., a mammalian chromosome, e.g., a mouse or a human chromosome, a yeast chromosome, an insect chromosome, a plant chromosome, a bacterial chromosome, e.g., an E. coli chromosome.

In one aspect, the invention provides CSM methods for generating diverse and ordered genomic libraries containing point mutations made by systematic mutagenesis of an entire chromosome. In one aspect, the invention provides methods for generating diverse and ordered E. coli libraries containing point mutations by systematic mutagenesis of the entire E. coli chromosome. The point mutations can be within precisely defined, overlapping segments. The mutations can be specific and directed, or, random, or both. These ordered libraries can be screened and selected for various properties. For example, the libraries can be used to “evolve” (modify) hosts. These library-modified hosts can be used to increase the expression of host or heterologous proteins, facilitate assay development, increase the production of biomolecules, or display other potentially desirable phenotypes and the like.

The CSM of the invention can comprise generating random or directed mutations over segments of an entire chromosome using various techniques. Randomly mutated, overlapping segments can be generated using, e.g., error-prone PCR. Error-prone PCR can generate random pools of mutations within defined, overlapping segments of a genome sequence. PCR primers can be designed to generate mutant pools representing all regions of a chromosome, e.g., the E. coli or human chromosome, or any other chromosome that has been completely or partially sequenced, at predefined intervals. Pools of mutated DNA segments are generated from each primer set.

These segments can be inserted precisely into a homologous chromosomal locus. They can be separately addressed. In one aspect, a markerless gene replacement technique is used. This can add the mutated, overlapping segments without addition of exogenous sequences.

The mutated genomic segments, the mutated chromosome and/or the libraries of the invention can be inserted (e.g., infected, transfected, transformed) into a host cell and expressed. Any evolved hosts displaying desirable phenotypes can be characterized, e.g., by sequencing with the corresponding primer set to determine the mutation that led to the phenotypic change. This linkage of functional and genomic information permits further engineering of hosts by the combination of advantageous mutations within a single organism as each iterative step does not leave sequences or markers that might interfere with subsequent recombinations.

The CSM methods of the invention are not limited to gene disruption or altering chromosomal architecture. They also comprise the introduction of point mutations into protein coding or transcriptional control regions. Thus, the CSM methods of the invention can introduce random or directed point mutations with the potential to alter protein function and/or gene expression. The CSM methods of the invention can incorporate random point mutations within a defined region of the chromosome without adversely affecting other areas of the chromosome without leading to a loss of host fitness or introduction of undesirable phenotypes. The CSM methods of the invention can preserve the ability to identify those mutations that generate desired phenotypes.

The CSM methods of the invention can further comprise screening of host cells into which the mutated genomic segments, the mutated chromosome and/or the libraries of the invention can be inserted. These mutant hosts can produce host varieties that are optimized for particular applications. The mutant hosts can be used to increase knowledge of the functional genomics of a host cell, e.g., an E. coli or human cell. The CSM methods of the invention can be used to collate information from the whole-scale evolution. This information can be used to targeting of homologous loci in other organisms. For example, information from the whole-scale evolution of E. coli can be used in other bacteria. This can facilitate the generation of optimized hosts for a variety of applications, including whole-scale evolution in other species, such as highly recombinogenic organisms as Bacilli, other bacteria, yeast, plant cells, human cells and the like.

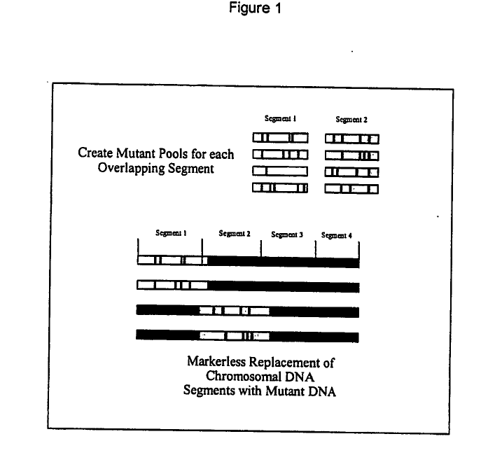

In one aspect, the methods of the invention generate mutant pools that represent defined regions of a chromosome, e.g., the E. coli chromosome, and incorporating the mutated alleles into the host's chromosome in a markerless, high-throughput fashion. The methods of the invention can further comprise mutagenesis to the entire chromosome, e.g., an E. coli chromosome, and generation and archiving of the mutant pools for identification of desired phenotypes. In one aspect, the CSM methods are carried out in the following steps, as summarized in FIG. 1:

1) Evaluate the ability of various error-prone PCR methodologies by routine screening to generate diverse mutant pools containing random point mutations;

2) Optimize the markerless replacement technique by routine screening to generate an efficient exchange of genetic material from the mutant allele to the chromosome;

3) Generate a large mutant pool for a specific region of the chromosome, e.g., the E. coli chromosome, using the methods optimized by routine screening as described above.

Generation of Mutations by PCR

In one aspect, polymerase chain reaction (PCR) is used to generate a population, e.g., a library of genomic segments, that contains random point mutations dispersed throughout the chromosome, genomic segment or gene. Other mutagenic techniques, such as GSSM or SLR, can be used systematically to alter one or more amino acid coding sequences, in one aspect, every amino acid, in a protein. In one aspect, the polypeptide coding sequence is altered such that each amino acid is altered to every other natural amino acid.

In one aspect, Taq DNA polymerase is used to practice the methods for the invention. In one aspect, biased nucleotide concentrations and/or the presence of manganese is utilized such that Taq DNA polymerase can incorporate mutations at a higher frequency. Exemplary methods for implementing PCR mutagenesis are described by Leung (1989) Technique 1(1):11-15; Cadwell (1992) PCRMeth. and Appl. 2:28-33; Vartanian (1996) Nucl. Acids. Res.,24(14):2627-2631. In one aspect, these protocols are utilized to generate mutant pools (e.g., libraries of genomic/chromosomal segments) during PCR amplification. These exemplary methods can be used for protein evolution studies. The methods of the invention can be applied to generate mutant pools of host genes. Another PCR-based mutagenesis strategy of the invention involves using a DNA polymerase that has been engineered to function with relaxed fidelity during amplification, see, e.g., Cline (2000) Strategies 13(4):157-161.

Introduction of Mutations into Chromosome by Markerless Recombination

After generating mutant pools, or libraries, of chromosomal/gene segments by, e.g., using PCR, a markerless replacement method is used to incorporate these mutant pools, or “mutant alleles,” into a chromosome, e.g., anE. coli chromosome, e.g., an MG1655 chromosome. Any markerless replacement method can be used, e.g., as described by Posfai (1999) Nucl. Acids Res., 27(22):4409-4415.

In one aspect, the approach is composed of two steps: a cointegration event followed by a resolution. An exemplary markerless replacement protocol is:

1) PCR products are cloned into a vector (pST98-KS) that is temperature-sensitive for replication, carries an antibiotic resistance gene (kpt) and also contains both the recognition site of meganuclease I-SceI and the encoding gene. Recombinant molecules are introduced into the E. coli host by electroporation and, following growth at non-permissive temperatures, cointegrants that result from homologous recombination into the genome between the mutant and the wild-type (wt) alleles, are selected by antibiotic selection.

2) Resolution of cointegrants by intramolecular recombination of the allele pair is forced by introduction of a unique double-strand break into the chromosome at the I-SceI recognition site by the vector-encoded I-SceI meganuclease gene whose expression is regulated by the addition of an inducer. Recombination between the alleles is necessary in order for cells to survive the introduction of the double strand break.

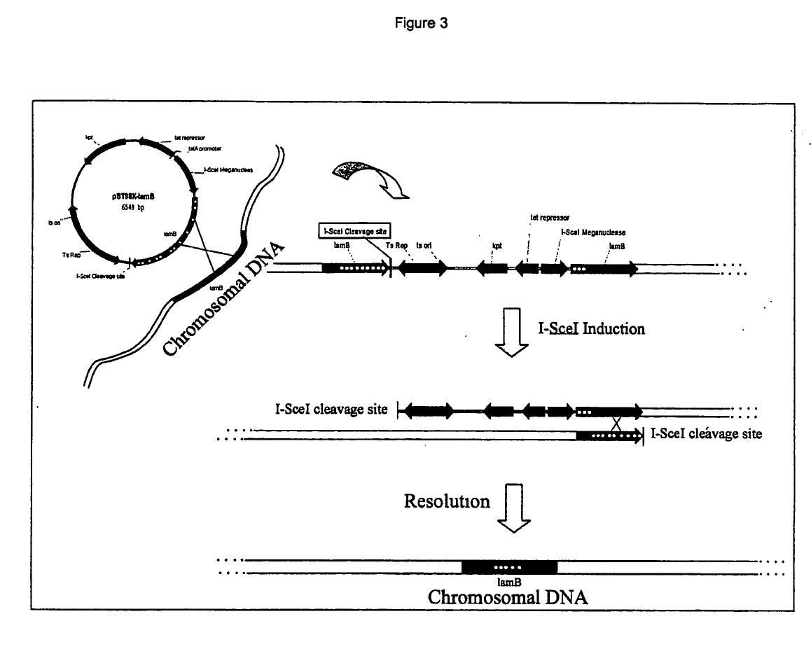

An overview of an exemplary markerless replacement method is illustrated in FIG. 3.

Resolution of the Cointegrant and Generation of the Markerless Replacement

In one aspect, following the cointegration of the mutated segment into a chromosome, the next step is the efficient resolution of the co-integrate and exchange of mutations into the host genome. In one aspect, the vector contains a restriction site for the intron-encoded meganuclease, e.g., a meganuclease I-SceI (see Example 2, below). Induced expression of this enzyme in the cointegrants can lead to the generation of a single double stranded break in a host chromosome. This recognition site does not occur naturally in an E. coli genome. In order to repair this double-stranded break, the duplicated sequences present in the cointegrate genome can be used as a substrate for recombination. This then results in the elimination of the plasmid vector sequences, including the antibiotic resistance gene, and the markerless exchange of some portion of the mutated segment into the chromosome, depending on where the cointegrative and resolving crossovers have taken place.

Mutagenesis of Entire Chromosome

In one aspect of the invention, an entire chromosome is randomly mutated or mutated in a directed fashion, or both, by the CSM methods of the invention. In one aspect, to accommodate the demands of generating mutant pools representing the entire 4,632 Kb E. coli genome, high throughput strategies are employed throughout the construction and screening steps. Robotic colony pickers and liquid replicates can be used to generate diverse pools in a high throughput manner from, e.g., 384-well or 1536 well plates. In one aspect, a GigaMatrix™ system is adapted to generate a diverse library. GigaMatrix™ can comprise about 100,000 individual wells housed within a typical plate footprint. GigaMatrix™ can be adapted for the construction steps. In this aspect, fewer plates are needed to generate a diverse library. In one aspect, following the markerless replacement portion of library construction, individual mutant hosts are recombined and stored as one mutant library representing that specific segment of E. coli.

Screening of Mutant Libraries for Desirable Phenotypes

In alternative aspects, the resulting library of mutated pools of nucleic acid segments (e.g., libraries or genomic/chromosomal segments), e.g., E. coli pools, are screened in several ways. First, if a host strains that shows decreased assay background is desired, the library can be arrayed into 384, 1536 or GigaMatrix™ plates and assayed for the a desired variant. Using sensitive fluorescent assay, the same procedure can be applied if an increase the activity of an endogenous gene (e.g., an E. coli gene) is desired. In specific cases, selection assays can also be utilized that would eliminate the need to array mutant host clones.

In one aspect, if an increase in the activity of a gene that is exogenously added to the host cell (e.g., E. coli) is desired, then the libraries (pools) are transfected or electroporated with a plasmid clone that contains the desired gene before screening or selection for the desired phenotypic variant. In order to preserve the ability to discover the link between particular mutations and altered phenotype, mutant pools for each segment can be transformed or transfected individually. In one aspect, electroporation is used with a 384-well electrode to transform 12, 384 well plates of mutant pools (libraries), assuming a 2 kb segment size and 50% overlap of segments, representing each segment of the chromosome for all exogenous genes assayed.

The discussion of the general products and methods given herein is intended for illustrative purposes only. Other alternative products, methods and embodiments will be apparent to those of skill in the art upon review of this disclosure.

The phrases “nucleic acid” or “nucleic acid sequence” can include an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

The term “saturation mutagenesis” or “GSSM” includes a method that uses degenerate oligonucleotide primers to introduce point mutations into a polynucleotide, as described in detail, below. The term “optimized directed evolution system” or “optimized directed evolution” includes a method for reassembling fragments of related nucleic acid sequences, e.g., related genes, and explained in detail, below. The term “synthetic ligation reassembly” or “SLR” includes a method of ligating oligonucleotide fragments in a non-stochastic fashion, and explained in detail, below.

The terms “vector” and “expression cassette” as used herein can be used interchangeably and refer to a nucleotide sequence which is capable of affecting expression of a nucleic acid, e.g., a mutated nucleic acid of the invention. Expression cassettes can include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids.

Generating and Manipulating Nucleic Acids

The methods of the invention modify nucleic acids, including all or parts of chromosomes, including entire genomes. The invention also includes methods for making new polypeptides and phenotypes using the modified nucleic acids of the invention. In practicing the invention, nucleic acid can be modified, i.e., mutated, by any method, e.g., polymerase chain reaction (e.g., error-based PCR), synthetic ligation reassembly, optimized directed evolution system and/or saturation mutagenesis.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, cloned (recombinant) or isolated nucleic acid, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440□3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

Vectors and Cloning Vehicles

In practicing the methods of the invention, the mutated nucleic acids segments can be inserted to a vector or a cloning vehicle before introduction into a homologous chromosome, including introduction into a host cell. Alternatively, selection markers inserted together with or independently of the mutated segment can be cloned into a vector or a cloning vehicle and inserted into a host cell with a mutated segment. Vectors and cloning vehicles can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The vector may comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the vectors contain one or more selectable marker genes to permit selection of host cells containing the vector (including or co-inserted into a host cell with a mutated gene segment). Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

A nucleic acid sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector may be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL Pharmacia). However, any other vector may be used as long as it is replicable and viable in the host cell.

Host Cells and Transformed Cells

The methods of the invention can comprise inserting a nucleic acid (e.g., a chromosome or a genome) modified (“mutated”) by the methods of the invention into a host cell. The methods can comprise screening the host cells for an altered genotype or phenotype. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

The mutated nucleic acid segments and/or vector(s) may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (see, e.g., Davis, et al., Basic Methods in Molecular Biology, (1986)).

Where appropriate, the engineered host cells can be cultured in conventional nutrient media modified as appropriate for activating promoters, selecting transformants or amplifying the genes or increasing homologous recombination. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

In one aspect, the nucleic acids or vectors made by the methods of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In one aspect of the invention one or more mutations are introduced into one or more nucleic acid (e.g., chromosomal or genomic) segments by amplification, e.g., by error-based polymerase chain reaction (PCR). Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Modification of Nucleic Acids

The invention provides methods for mutating a nucleic acid sequence comprising providing segments of a chromosome or part of a chromosome and introducing one or more mutations into one or more of the segments. Any method can be used to introduce the mutation, either randomly, non-randomly, or both. These methods can be repeated or used in various combinations to generate one or more mutations into one or more of the segments. These methods also can be repeated or used in various combinations. In another aspect, the genetic composition of a cell is altered by, e.g., modification of a homologous gene ex vivo by a method of the invention followed by its reinsertion into a cell.

Any method can be used to introduce the mutation, either randomly, non-randomly, or both. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols used in the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional protocols used in the methods of the invention include those discussed in U.S. Pat. No.5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Protocols that can be used to practice the invention (providing details regarding various diversity generating methods) are described, e.g., in U.S. patent application Ser. No. 09/407,800, “SHUFFLING OF CODON ALTERED GENES” by Patten et al. filed Sep. 28,1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18,2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-stochastic, or “directed evolution,” methods include, e.g., saturation mutagenesis (GSSM), synthetic ligation reassembly (SLR), or a combination thereof are used to modify the nucleic acids of the invention to generate mutated nucleic acid segments. Polypeptides encoded by these modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM

In one aspect of the invention, non-stochastic gene modification, a “directed evolution process,” is used to generate mutated sequences. Variations of this method have been termed “gene site-saturation mutagenesis,” “site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

The methods of the invention include use of non-stochastic gene modification system termed “synthetic ligation reassembly,” or simply “SLR,” a “directed evolution process,” to generate mutated sequences of the invention. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

The methods of the invention also use non-stochastic gene modification system termed “optimized directed evolution system” to generate mutated sequences of the invention. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Iterative Processes

In practicing the invention, these processes can be iteratively repeated. For example a nucleic acid (or, the nucleic acid) responsible for an phenotype is identified, re-isolated, again modified, re-tested for activity. This process can be iteratively repeated until a desired phenotype is engineered. For example, an entire biochemical anabolic or catabolic pathway can be engineered into a cell, including proteolytic activity.

Similarly, if it is determined that a particular oligonucleotide has no affect at all on the desired trait, it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In vivo shuffling of molecules can be used in methods of the invention. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In one aspect, the invention provides a method for producing a hybrid polynucleotide from at least a first polynucleotide and a second polynucleotide. The invention can be used to produce a hybrid polynucleotide by introducing at least a first polynucleotide and a second polynucleotide which share at least one region of partial sequence homology into a suitable host cell. The regions of partial sequence homology promote processes which result in sequence reorganization producing a hybrid polynucleotide. The term “hybrid polynucleotide”, as used herein, is any nucleotide sequence which results from the method of the present invention and contains sequence from at least two original polynucleotide sequences. Such hybrid polynucleotides can result from intermolecular recombination events which promote sequence integration between DNA molecules. In addition, such hybrid polynucleotides can result from intramolecular reductive reassortment processes which utilize repeated sequences to alter a nucleotide sequence within a DNA molecule.

Producing Sequence Variants

The methods of the invention introduce one or more mutations into one or more of nucleic acid segments. The nucleic acids can be altered by any means, including, e.g., random or stochastic methods, or, non-stochastic, or “directed evolution,” methods, as described above.

Mutations can be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, mutations may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP , 0.2 mM DATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Mutations may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating mutations is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating mutations is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:1 in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Mutations may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Mutations may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate mutations. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, mutations are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, mutations are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

Capillary Arrays

In one aspect the methods of the invention can be practiced in a reaction chamber such as a capillary array. The methods of the invention also can be practiced in whole or in part using capillary arrays. The product of manufacture and the methods of the invention can comprise a capillary array such as the GIGAMATRIX™, Diversa Corporation, San Diego, Calif. See, e.g., WO 0138583. In practicing the products and methods of the invention, reagents or polypeptides, e.g., enzymes, such as PCR polymerases, and the like, can be immobilized to or applied to an array, including capillary arrays. Capillary arrays provide another system for holding and screening reagents, enzymes, and products of reactions. The apparatus can further include interstitial material disposed between adjacent capillaries in the array, and one or more reference indicia formed within of the interstitial material. High throughput screening apparatus can also be adapted and used to practice the methods of the invention, see, e.g., U.S. patent application Ser. No. 20020001809.

Whole Cell-Based Methods

The CSM processes of the invention can be practiced in whole or in part in a whole cell environment. The invention also provides for whole cell evolution, or whole cell engineering, of a cell to develop a new cell strain having a new genotype or phenotype. This can be done by modifying the genetic composition of the cell by the methods of the invention, where the genetic composition is modified by addition to the cell of a modified nucleic acid segment or chromosome or genome made by a method of the invention. See, e.g., WO0229032; WO0196551.

The host cell for the “whole-cell process” may be any cell known to one skilled in the art, including prokaryotic cells, eukaryotic cells, such as bacterial cells, fungal cells, yeast cells, mammalian cells, insect cells, or plant cells.

To detect the production of an intermediate or product of the methods of the invention, or a new phenotype, at least one metabolic parameter of a cell (or a genetically modified cell) is monitored in the cell in a “real time” or “on-line” time frame by Metabolic Flux Analysis (MFA). In one aspect, a plurality of cells, such as a cell culture, is monitored in “real time” or “on-line.” In one aspect, a plurality of metabolic parameters is monitored in “real time” or “on-line.”

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including the:
identity of all pathway substrates, products and intermediary metabolites
identity of all the chemical reactions interconverting the pathway metabolites, the stoichiometry of the pathway reactions,
identity of all the enzymes catalyzing the reactions, the enzyme reaction kinetics,
the regulatory interactions between pathway components, e.g. allosteric interactions, enzyme-enzyme interactions etc,
intracellular compartmentalization of enzymes or any other supramolecular organization of the enzymes, and,
the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available. Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization.

Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc. Any aspect of metabolism or growth can be monitored.

Monitoring Expression of a Polypeptides, Peptides and Amino Acids

In one aspect of the invention, new phenotypes are monitored in a cell after introduction of a modified nucleic acid. In one aspect of the invention, an engineered phenotype comprises increasing or decreasing the expression of a polypeptide or generating new polypeptides in a cell. Production of peptides and increased or decreased expression of new or altered polypeptides can be traced by use of a fluorescent polypeptide, e.g., a chimeric protein comprising an enzyme used in the methods of the invention.

Polypeptides, reagents and end products also can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Polypeptides of a cell can be measured using a protein array.

### EXAMPLES

### Example 1

### Assessment of Frequency and Distribution of PCR Mutations

The following example describes exemplary methods of the invention.

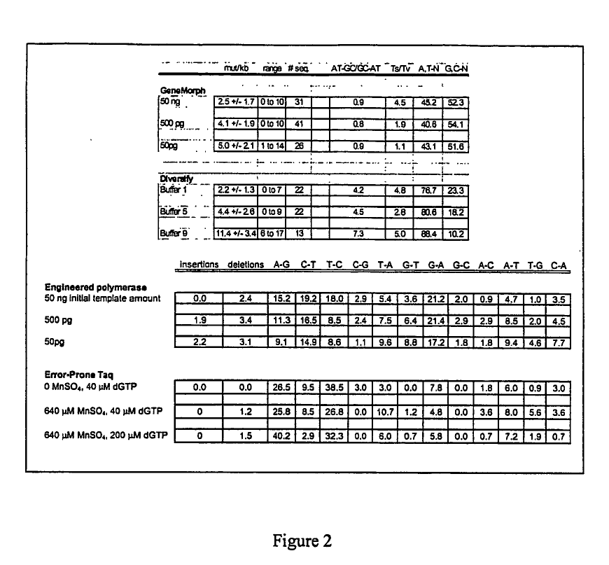

Three target genes (b1625, rcsB and lamB) were amplified from E. coli MG1655 genomic DNA using either the error-prone Taq PCR method or using a polymerase engineered to frequently introduce mutations. PCR products were then cloned and several clones from each target and frequency level were sequenced to determine the type and number of mutations introduced with the different methods. FIG. 2 shows a table comparing exemplary PCR based mutagenesis methods used in the methods of the invention.

In one aspect, to determine the best method of generating diverse mutant pools, E. coli gene targets are selected and amplified using at least two separate PCR mutagenesis strategies. Under certain reaction conditions, such as biased nucleotide concentrations and/or the presence of manganese, Taq DNA polymerase can incorporate mutations at a higher frequency. Exemplary methods for implementing PCR mutagenesis are described by Leung (1989) Technique 1(1): 11-15; Cadwell (1992) PCR Meth. and Appl. 2:28-33; Vartanian (1996) Nucl. Acids. Res.,24(14):2627-2631. Each selected target can be amplified using conditions that generate different mutation frequencies with each system. With Taq-based error-prone PCR, mutations can be introduced at different frequencies by altering the reaction conditions, including, e.g., manganese and nucleotide concentration. Using an engineered polymerase, the mutation frequency can be altered by changing the amount of starting template, as the overall mutation frequency is a function of the polymerase error rate and the number of amplification steps.

In one aspect, products resulting from PCR amplification are cloned. Representative samples can be sequenced to determine the type, position, and frequency of mutations introduced. Statistical analyses can be applied to determine both the diversity of the pools generated using each approach and the number required to generate statistically relevant data. The data from each system can be compared to determine the best method to generate diverse mutant pools of host genes. An average of five to seven nucleotide changes per kb can be targeted. Enzymes displaying desired phenotypic alterations can be isolated from moderately sized libraries with mutation frequencies in this range; see, e.g., Daugherty (2000) Proc. Natl. Acad. Sci. USA 97:2029-2034; Christians (1996) Proc. Natl. Acad. Sci. USA 93:6124-6128; Martinez (1996) EMBO J. 15:1203-1210, for exemplary protocols to be incorporated into the methods of the invention.

In one aspect, PCR product sizes of approximately 2 kb are generated initially for several reasons. First, a 2 kb fragment is small enough to be consistently generated with error-prone PCR protocols that are designed to provide conditions under which the polymerase works ineffectively. Second, a 2 kb fragment is small enough to be sequenced quickly to characterize mutations that produce desired phenotypic variants. Third, only 4,300 fragments are needed to cover the entire E. coli chromosome (with 50% overlap between PCR fragments). The 2 kb size therefore is a compromise between the number of segment pools necessary to cover the chromosome and preserving the ability to quickly line genotypic and phenotypic information.

The error-prone Taq method shows more mutational bias than the engineered polymerase with adenine or thymine substitutions accounting for approximately 80% of the mutations introduced. This is consistent with previously published results. An engineered polymerase can show less mutational bias, with guanine or cytosine substitution only slightly favored over others (55%). Both methods were able to consistently introduce mutations with frequencies that could be altered as expected, although more clones must be sequenced to obtain a more accurate measure of mutation frequency and pool diversity. Of the clones chosen for sequencing, none contained the same mutation in the same position in the amplified product.

### Example 2

### Co-Integration of Mutations into Chromosome

The following example describes an exemplary method of the invention for co-integrating mutations into chromosomes.

In one aspect, to determine optimal conditions for the co-integration and subsequent resolution of PCR-generated mutations into the chromosome, a single mutated fragment about 2 kb in size, containing the E. coli lamB gene, is used. This fragment can be chosen from those cloned and sequenced during the comparison of PCR mutagenesis methods, as described above, to have a relatively large number of evenly distributed mutations (n>10 with an average of 1 substitution per 200 nucleotides (nt)) so that the location of recombinational intervals can be readily mapped. The distribution of the point mutations that ultimately end up in the chromosome after markerless recombination can be important to determine because it may dictate the amount of overlap necessary in the PCR generated fragments to insure that all portions of the chromosome contain point mutations.

In one aspect, the selected lamB mutant allele is subcloned into the recombination vector, pST98-KS, and transformed into E. coli MG1655. Transformants can be grown on agar plates overnight under conditions permissive for plasmid growth (30° C.). To obtain a rough measure of recombination efficiency based on the number of wells that grow after temperature selection, colonies can be arrayed by an automated colony picker into 384-well microtiter plates (containing the appropriate growth media) and incubated overnight at 30° C. to increase cell number under permissive conditions. Aliquots of the 384 cultures can be sequentially transferred to new microtiter plates containing growth media and antibiotic and incubated at 42° C., a temperature non-permissive for autonomous plasmid replication.

A parallel experiment can be performed with a negative control, using colonies transformed with the vector alone that contains no homologous chromosomal sequence to mediate cointegration. In one aspect, sequential transfers are carried out until no growth of the negative control is observed, while still observing growth in wells containing cloned mutant alleles. In one aspect, the number of wells containing cloned mutant alleles that grow after successive rounds at 42° C. is determined. As the number of recombination events per well may increase if more inoculum is used, several different inoculum volumes can be tested and compared to determine the optimal amount to transfer from plate to plate to insure the diversity of the pool is not compromised.

In one aspect, in order to determine that recombination is occurring at the homologous locus in the E. coli chromosome and the timing of the co-integration events, PCR is performed on DNA from cells sampled at various time points during the transfers from permissive to non-permissive temperatures using primers external to the chromosomal lamB locus. In one aspect, to insure that recombination is occurring at the appropriate homologous location in the chromosome, inverse PCR or vectorette PCR is used to identify aberrant sites of integration. In one aspect, inverse PCR is utilized using overlapping primers internal to the vector sequence and circular fragments of chromosomal DNA that has been digested with a restriction enzyme and self-ligated as template. Vectorette PCR can also provide a way to map the DNA flanking the vector using a vector specific primer in conjunction with a second primer complementary to a priming site that is ligated to a DNA end generated from restriction digestion of the chromosomal DNA prior to PCR; an exemplary protocol is described in Allen (1994) PCR Methods Appl. 4(2):71-75.

Assessment of Recombination Efficiency and Temperature Selection in Liquid Culture as Determined by PCR

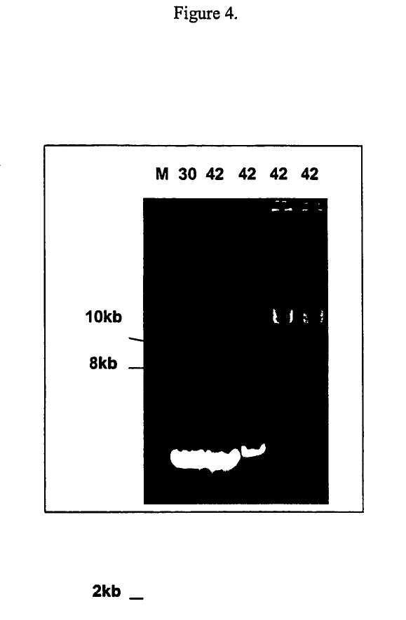

Colonies transformed with the pST98-KS vector containing a 1.7 kb mutant version of lamB (# mutations=12) were cultured at 30° C. for 18 hours in LB with antibiotic. Ten ul of culture was used to start a new liquid culture in the same media. The new culture was grown at 42° C. overnight. The culture was passed in this manner for an additional three rounds. Genomic and plasmid DNA were purified from each culture every day. PCR amplifications were then performed to determine the relative amounts of wild-type and recombined lamB present in the chromosome at various temperature stages. PCR primers were designed to amplify only the chromosomal lamB sequence and were positioned external to this locus. Five microliters of each PCR reaction was run on a 1% agarose, 1×TAE gel. Results from this experiment are shown in FIG. 4.

If wild type lamB locus is present, a band of 2.3 kb should be amplified. If the plasmid recombines into the chromosome at the lamB locus, a band of 8.65 kb should be amplified. The results (FIG. 4) show that the presence of the wild type version of the locus within cells in the culture decreases as the temperature is increased to 42° C. However, it takes several cycles of growth at 42° C. to significantly decrease the amount of wild type lamB and concomitantly increase the amount of plasmid that has recombined into the appropriate locus of the chromosome. The results also show that a high percentage of the cells are recombining with plasmid, as very little native genomic DNA band remains after three rounds at 42° C.

Resolution of the Cointegrant and Generation of the Markerless Replacement

In one aspect, following the co-integration of the mutated segment (cloned in pST98-KS) into the chromosome, the next step is the efficient resolution of the co-integrate and exchange of mutations into the host genome. pST98-KS contains a restriction site for the intron-encoded meganuclease I-SceI, induced expression of this enzyme in the co-integrants. This can lead to the generation of a single double stranded break in the host chromosome (this recognition site does not occur naturally in the E. coli genome). In order to repair this double-stranded break, E. coli can use the duplicated sequences present in the co-integrate genome as a substrate for recombination. This then results in the elimination of the plasmid vector sequences (including the antibiotic resistance gene) and the markerless exchange of some portion of the mutated segment into the chromosome, depending on where the cointegrative and resolving crossovers have taken place. A portion of the resulting population may regenerate the wild-type sequence.

After cultures have grown and transferred through an appropriate number of cycles at 42° C., aliquots will be replicated into a fresh 384-well plate containing growth media without kanamycin and the inducer of meganuclease expression, chlortetracycline (from the tet promoter). At various times, aliquots will be removed and plated onto either LB or LB/Kan plates to monitor the progression of the loss of antibiotic resistance (as a result of resolution of the allele pair and loss of the vector sequences). This will establish the length of time required for growth in the presence of inducer necessary to achieve complete cleavage and resolution of the allelic pair in all cells. After the appropriate amount of time in inducer, aliquots from a number of wells will be plated on LB-agar and incubated overnight to select individual clones for further analysis. High fidelity PCR amplifications will then be performed for 50 clones from each plate using primers that are positioned external to each gene to generate template for sequencing. Sequencing will be used to determine the distribution of mutations introduced.

Resolution of the Allele Pair Following Meganuclease Induction

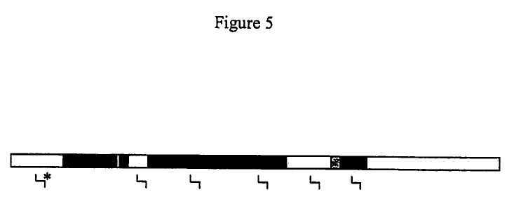

The mutated E. coli lamB gene fragment (described above), cloned into pST98-KS was transformed into E. coli MG1655 cells. Colonies were inoculated and cultured at 30° C. to permit the formation of the recombination intermediate. Cultures were then transferred through four rounds of growth at 42° C. (determined as described above) after which meganuclease expression was induced by transferring cultures to LB media with 20 ug/ml of heat-inactivated chlortetracycline. Cultures were grown at 30° C. overnight and aliquots were plated on LB and LB/Kan plates. No colonies grew on plates containing kanamycin while thousands were produced on LB-only plates. Twenty-five of these colonies were used as template for PCR and the products were sequenced to map recombination sites. Approximate recombination sites for the clones are indicated in FIG. 5 (an illustration of the gross recombination site mapping). Twenty-three of clones contained mutations that were originally present in the mutant allele, while only two of the clones were found to retain the wild type lamB sequence.

In FIG. 5, regions bounded by the twelve substitutions in the lamB mutant allele are differentially colored. Sequencing detects native or mutant sequence at each of the twelve mutant positions. When the sequence switches from mutant to native or native to mutant in the host chromosome following markerless replacement, a crossover must have occurred in the previous segment. Regions where crossovers occurred in the 25 sequenced clones are indicated. When recombination takes place outside the mapped regions (*) all twelve mutations are present.

Creation of a Mutant Pool of Host Cells that Contain Point Mutations Dispersed Throughout a Specific Chromosomal Segment

In one aspect, once the relevant parameters have been determined using a specific mutant allele, saturation mutagenesis is performed on a defined region of the E. coli chromosome. In order to insure reasonably complete mutagenesis of a given 2 kb interval, it is estimated that at least 100,000 individual cloned fragments are need to be taken through the CSM procedure of the invention. Enzymes with desired phenotypes can be found in libraries of 100,000 members or smaller. If the individual, mutagenized fragments are kept separate throughout the entire CSM procedure, it may be necessary to use ultra-high throughput procedures to carry out these experiments. For example, if separate fragments are to be arrayed during the construction, approximately 250, 384-well plates can be used to generate a 100,000 member library for a given segment. Combining the cloned fragments of a given segment prior to the co-integration and resolution step can reduce the magnitude of materials and effort required, however, this may limit the diversity seen in the final mutant population if, for instance, some recombination events during the co-integration occur earlier than others and the population becomes biased toward these early events.

To determine the extent to which combining individual clones will be possible without compromising diversity, the following experiment can be carried out. Mutagenized lamB containing fragments can be generated by PCR and cloned into the pST98-KS recombination vector. Individual clones can be selected and the fragment sequenced to identify the mutations present in each. A plurality of clones, e.g., about fifty (50) clones, can be selected, with the criteria that the composition of mutations is unique to each fragment. Five mixes of increasing complexity can be generated to contain, e.g., about 2, 5, 10, 25 and 50 members. Each pool can be inoculated in triplicate into 384-well plates, taken through the optimized CSM procedure, and plated onto LB-agar minus kanamycin. In one aspect, to characterize the diversity of the resulting library, 50 colonies from each well are grown and templates prepared for sequencing (750 total sequences).

By determining the distribution of the parental contribution (since each mutation will be attributable to a single parent) to the final composition of the mutants, it is possible to determine the maximum pool size that most effectively maintains the diversity present in the original mutations. For example, if it is determined that a pool size of about 50 maintains a reasonable level of diversity, this will decrease the number of 384-well plates required from 250 to 5 for a given segment of the chromosome. It may be possible to increase the pool sizes higher than 50, although analysis of the diversity of events will become increasingly difficult.

If the experiment described above shows that competition between alleles does in fact limit diversity when multiple alleles are combined and subjected to CSM together, then stimulating homologous recombination may provide a mechanism to address the problem. Making homologous recombination more efficient may increase the number of recombination events that occur in early growth stages and minimize the effects of over-competition. Thus, in one aspect, the methods of the invention comprise cloning and expressing recA on the recombination plasmid to increase homologous recombination. However, the E. coli recA gene could serve as another chromosomal recombination site. It may be possible to decrease the likelihood that the chromosomal version might act as an alternate recombination spot with the plasmid by cloning a non-E. coli version of recA that has less homology with the E. coli version, but has been shown to both increase homologous recombination when expressed in E. coli and not to induce the SOS response. Thus, in one aspect, the invention comprises cloning and expressing a non-E. coli version of recA.

In one aspect, expressing recA from the recombination plasmid is done in conjunction with using the markerless replacement technique with RecA-strains of E. coli, such as those commonly used for cloning and expression experiments.

Inducing endogenous RecA activity can also increase homologous recombination. Thus, in one aspect, the methods of the invention comprise inducing endogenous RecA activity. In alternative aspects, this strategy is accomplished in several ways. In one aspect, linear fragments are cotransformed into MG1655 with those ligated into the recombination vector. It has been shown that the presence of linear DNA induces homologous recombination.

Homologous recombination has also been shown to be stimulated by the introduction of mismatched DNA into a cell, such as could be generated if the mutant pool were denatured and re-annealed prior to cloning. Thus, in one aspect, the methods of the invention comprise introduction of mismatched DNA into a cell.

It also may be possible to induce endogenous RecA activity by denaturing the plasmids that contain mutated inserts and re-annealing them in the presence of excess linear PCR product in an attempt to form triplex structures. Triplex structures are known to increase homologous recombination. Thus, in one aspect, the methods of the invention comprise introducing triplex structures or inducing the formation of triplex structures.

Homologous recombination may also be increased by creating an area of single strandedness to which the RecA filament can more efficiently bind. This can be done by denaturing the cloned mutated segment (“mutated allele”) in the presence of vector alone and then re-annealing the two together. This may create a looping out of the homologous mutated allele prior to electroporation, thereby providing a more efficient recombination substrate in the cell. Thus, in one aspect, the methods of the invention comprise creating an area of single strandedness to which the RecA filament can more efficiently bind.

A number of embodiments of the invention have been described. Nevertheless, it will be understood that various modifications may be made without departing from the spirit and scope of the invention. Other embodiments are within the scope of the following claims.

1. A method for mutating a nucleic acid sequence comprising the following steps
(a) providing segments of a chromosome or part of a chromosome;
(b) introducing one or more mutations into one or more of the segments; and
(c) reinserting the mutated segments into a homologous chromosome with a markerless gene replacement technique.

2. The method of claim 1, wherein the chromosome of step (a) comprises an entire genome.

3. The method of claim 1, wherein the chromosome of step (a) comprises an entire chromosome.

4. The method of claim 1, wherein the chromosome is a bacterial chromosome.

5. The method of claim 4, wherein the bacterial chromosome is an E. coli chromosome.

6. The method of claim 1, wherein the chromosome is a yeast, plant, insect or mammalian chromosome.

7. The method of claim 6, wherein the mammalian chromosome is a human chromosome.

8. The method of claim 1, wherein the segments are overlapping.

9. The method of claim 8, wherein the segments are overlapping by 2, 3, 4, 5, 6, 7, 8, 9, 10, 11, 12, 13, 14, 15, 16, 17, 18, 19, 20, 25, 30, 35, 40, 45, 50 or 55 base pairs.

10. The method of claim 1, wherein the segments are not overlapping.

11. The method of claim 1, wherein the segments are about 5, 10, 20, 30, 40, 50, 60, 70, 80, 90, 100, 150 or 200 base pairs in length.

12. The method of claim 1, wherein the segments are about 250, 300, 350, 400, 450, 500, 550, 600, 650, 700, 750, 800, 850, 900, 950, 1000, 1500, 2000, 2500, 3000, 3500, 4000, 4500, 5000 base pairs in length.

13. The method of claim 1, wherein the segments are between about 10 and 500,000 base pairs, or, between about 50 and 250,000 base pairs, in length.

14. The method of claim 1, wherein the mutations are randomly introduced.

15. The method of claim 1, wherein the mutations are non-randomly introduced.

16. The method of claim 1, wherein the mutations are introduced by polymerase chain reaction (PCR).

17. The method of claim 16, wherein the polymerase chain reaction (PCR) is error-prone polymerase chain reaction (PCR).

18. The method of claim 1, wherein mutated segments can comprise a GSSM library or a TGR (Tunable Gene Reassembly) library at one or more segments.

19. The method of claim 1, wherein the mutations are introduced into polypeptide open reading frames.

20. The method of claim 1, wherein the mutations are introduced into non-coding sequences.

21. The method of claim 1, wherein 1%, 5%, 10%, 15%, 20%, 25%, 30%, 35%, 40%, 45%, 50%, 55%, 60%, 65%, 70%, 75%, 80%, 90%, 95% or 100% of a genome or a chromosome is mutagenized.

22. The method of claim 1, wherein the mutated nucleic acid segments are introduced to a homologous chromosome in vitro.

23. The method of claim 1, wherein the mutated nucleic acid segments are introduced to a homologous chromosome in vivo.

24. The method of claim 23, further comprising inserting the mutated segments into a host cell comprising the homologous chromosome.

25. The method of claim 24, wherein the mutated nucleic acid segments introduced into the host cell with a selectable marker.

26. The method of claim 25, wherein the selectable marker is an antibiotic selection marker.

27. The method of claim 26, wherein the antibiotic selection marker is ampicillin, a beta lactam antibiotics, a semisynthetic penicillin, amoxycillin, ampicillin, methicillin, carbenicillin, tetracycline, chloramphenicol, a macrolide, erythromycin, an aminoglycoside, streptomycin, nalidixic acid, quinoline, rifamycin, sulfonamide, Gantrisin or Trimethoprim.

28. The method of claim 24, further comprising selecting a host cell comprising an altered genotype.

29. The method of claim 24, further comprising selecting a host cell comprising an altered phenotype.

30. The method of claim 24, further comprising inducing endogenous RecA activity in the host cell.

31. The method of claim 24, further comprising inducing or increasing homologous recombination in the host cell.

32. The method of claim 31, wherein homologous recombination is increased in the host cell by introducing mismatched DNA into the cell.

33. The method of claim 31, wherein homologous recombination is increased in the host cell by denaturing a cloned mutated segment in the presence of a vector alone and then re-annealing the two together before introduction into the cell.

34. The method of claim 31, wherein homologous recombination is increased in the host cell by introducing triplex structures or inducing the formation of triplex structures in the cell.

35. The method of claim 24, wherein the mutated segments are cloned into a vector before insertion into the host cell.

36. The method of claim 24, wherein the mutated segments are inserted into the host cell by electroporation.

37. The method of claim 24, wherein the mutated segments are inserted into the host cell by infection or transfection.

38. The method of claim 17, wherein the error-prone polymerase chain reaction (PCR) is a Taq-based error-prone PCR.

39. A library of mutated nucleic acid sequence made by a method comprising the following steps:
(a) providing segments of a chromosome or part of a chromosome;
(b) introducing one or more mutations into one or more of the segments; and
(c) reinserting the mutated segments into a homologous chromosome with a markerless gene replacement technique.

40. A cell comprising a library of mutated nucleic acid sequences, the library made by a method comprising the following steps:
(a) providing segments of a chromosome or part of a chromosome;
(b) introducing one or more mutations into one or more of the segments; and
(c) reinserting the mutated segments into a homologous chromosome with a markerless gene replacement technique.
